(19) United States
(12) Patent Application Publication
(10) Pub. No.: US 2024/0345071 A1
(43) Pub. Date: Oct. 17, 2024

(54) COMPOSITION EVALUATION METHOD

(71) Applicant: Konica Minolta, Inc., Tokyo (JP)

(72) Inventors: MAYUKA KABASAWA, hamura-shi, Tokyo (JP); HIROSHI KITA, Hachioji-shi, Tokyo (JP)

(21) Appl. No.: 18/579,157

(22) PCT Filed: Mar. 15, 2022
(86) PCT No.: PCT/JP2022/011675

§ 371 Date: Jan. 12, 2024

(30) Foreign Application Priority Data
Aug. 2, 2021 (JP) 2021-126668

(51) Int. Cl. G01N 33/53; G01N 33/543

(52) U.S. Cl. CPC G01N 33/5308; G01N 33/54313

(57) ABSTRACT

An object of the present invention is to provide a composition evaluation method capable of grasping the state of a composition by a simple method without separating an object. A composition evaluation method that solves the above object includes the steps of: mixing a composition containing one or more types of objects and a labeling substance having an interaction portion and a fluorescence-emitting portion to prepare a mixture; acquiring a fluorescence fingerprint for the mixture; and evaluating a state of the composition based on the fluorescence fingerprint, wherein the interaction portion has a nucleic acid structure capable of interacting with each of a plurality of types of the objects and/or a nucleic acid structure capable of interacting with a plurality of positions of each of one or more types of the objects.

### TECHNICAL FIELD

The present invention relates to a composition evaluation method.

### BACKGROUND ART

In recent years, while COVID-19 virus infection is raging, an almost unprecedented mRNA vaccine has been newly developed, and an efficacy ratio exceeding that of a conventional vaccine has been confirmed. In response to this, mRNA preparations are expected to be developed in various applications and further developed. However, mRNA is generally unstable, and is easily degraded by RNA catabolic enzyme, namely, RNase that exists in the air. In view of the above, attempts have been made to protect mRNA vaccines with lipid nanoparticles or the like. However, when the mRNA vaccine is stored for a long period of time or environmental changes occur during transportation, the lipid nanoparticles may aggregate, or the mRNA contained in the nanoparticles may leak out and deteriorate. Therefore, the mRNA vaccine is difficult to store and transport. Due to such characteristics of mRNA, it is expected that a simple inspection capable of determining whether or not a vaccine can be used and calculating the degree of deterioration immediately before use is required from the viewpoint of quality assurance.

In general, measurement by real-time PCR, capillary gel electrophoresis, or the like is used for verification of RNA, but such measurement methods require purification of RNA, and there is a concern about degradation at the time of handling.

On the other hand, in the clinical field, a method for quantifying the amount of various analytes by signal intensity of a fluorescent dye is known. However, in the evaluation method, when the excitation wavelengths and the maximum wavelengths of a plurality of identifiers (fluorescent dyes) overlap with each other, it is very difficult to distinguish between them. Therefore, the number of identifiers (fluorescent dyes) that can be used at a time is limited, and it is difficult to apply the method in a case where a large number of components are mixed in the analyte.

Here, the fluorescence fingerprint technology, which has been difficult to utilize due to excessive data, can be effectively utilized as inductive data by using artificial intelligence (AI). The fluorescence fingerprint (or excitation-emission matrix) measurement is a brute-force measurement that measures the intensity of fluorescence while changing both the wavelength of excitation light and the wavelength of fluorescence to be observed. The fluorescence fingerprint technology is used for quality evaluation of food and the like. The quality can be evaluated by, for example, analyzing various fluorescence signals emitted from each component originally contained in food.

In recent years, attempts have been made to develop the fluorescence fingerprint technology also in quantification by the signal intensity of the fluorescent dye described above. For example, Patent Literatures 1 and 2 describe that an object is modified with, for example, a fluorescent labeling substance that specifically acts only on the object, and the object is evaluated by fluorescence fingerprint.

On the other hand, various fluorescent labeling substances that specifically act on an object have also been developed. For example, oligonucleotide labeling substances containing a fluorescent group between adjacent nucleotides have also been developed (for example, Patent Literature 3). When the labeling substance interacts with a nucleic acid that is an object, the labeling substance emits a detectable fluorescence signal.

### CITATION LIST

### Patent Literature

Patent Literature 1: JP 2020-201202 A
Patent Literature 2: JP 2012-507710 T
Patent Literature 3: WO 2010/001902 A1

### SUMMARY OF INVENTION

### Technical Problem

Here, it is conceivable to apply the fluorescence fingerprint technology as described in Patent Literatures 1 and 2 to the evaluation of the degree of deterioration of the mRNA vaccine. However, when a labeling substance that specifically interacts with an object is used as in Patent Literatures 1 and 2, the labeling substance does not interact with a deteriorated product. Therefore, although the presence or absence of the object can be confirmed, the deterioration state of the object cannot be evaluated. On the other hand, it is also conceivable to separately prepare another labeling substance that specifically acts on the degraded product. However, since various degraded products and the like are usually generated by deterioration, it is necessary to prepare a large number of labeling substances corresponding to each deteriorated product. That is, the preparation requires a large amount of cost and time, and is not realistic. Furthermore, in the method, when the object changes to an unexpected structure, the component cannot be detected.

An object of the present invention is to provide an evaluation method capable of grasping the state of a composition containing one or more types of objects by a simple method without separating the objects.

### Solution to Problem

The present invention provides the following composition evaluation method.

A composition evaluation method including the steps of: mixing a composition containing one or more types of objects and a labeling substance having an interaction portion and a fluorescence-emitting portion to prepare a mixture; acquiring a fluorescence fingerprint for the mixture; and evaluating a state of the composition based on the fluorescence fingerprint, wherein the interaction portion has a nucleic acid structure capable of interacting with each of a plurality of types of the objects and/or a nucleic acid structure capable of interacting with a plurality of positions of each of one or more types of the objects.

The present invention also provides the following composition evaluation method.

A composition evaluation method including the steps of: mixing a composition containing one or more types of objects and a labeling substance having an interaction portion and a fluorescence-emitting portion to prepare a mixture; irradiating the mixture with excitation light having a predetermined wavelength and measuring fluorescence emitted from the mixture; and evaluating a state of the object from a result of the measurement, wherein the interaction portion of the labeling substance has a nucleic acid structure capable of interacting with each of a plurality of types of the objects and/or has a nucleic acid structure capable of interacting with a plurality of positions of each of the objects, and a wavelength of the excitation light is specified from a state estimation model created by preparing a plurality of samples having different states of the composition from each other, mixing the labeling substance with each of the plurality of samples to prepare a plurality of standard mixtures, acquiring a standard fluorescence fingerprint for each of the plurality of standard mixtures, and subjecting a state of the composition in each of the plurality of samples and each of the standard fluorescence fingerprints to machine learning.

### Advantageous Effects of Invention

According to the composition evaluation method of the present invention, it is possible to grasp the state of a composition containing one or more types of objects by a simple method without separating the objects.

### BRIEF DESCRIPTION OF DRAWINGS

FIGS. 1A and 1B are graphs showing an example of a fluorescence fingerprint.

FIG. 2 is a diagram illustrating a flow of a first embodiment.

FIG. 3 is a diagram illustrating a flow of a modification of the first embodiment.

FIG. 4 is a diagram illustrating a flow of a method for preparing a state estimation model.

FIG. 5 is a diagram illustrating a flow of a second embodiment.

FIG. 6 is a diagram illustrating a flow of determining a predetermined wavelength used in the second embodiment.

### DESCRIPTION OF EMBODIMENTS

Hereinafter, two embodiments of the present invention will be described in detail. However, the present invention is not limited to these embodiments.

### 1. First Embodiment

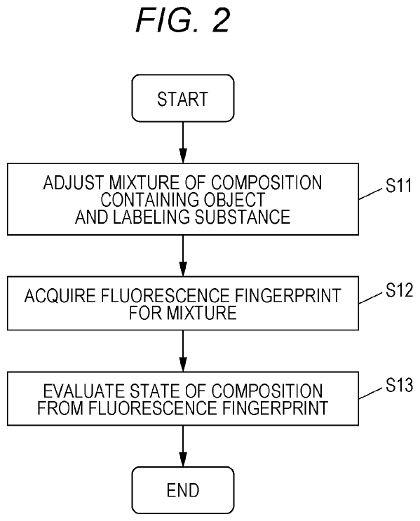

The flow of the composition evaluation method of the present embodiment is shown in FIG. 2. In the composition evaluation method of the present embodiment, a step S11 (hereinafter, also referred to as a “mixture preparation step”) of mixing a composition containing one or more types of objects and a labeling substance having an interaction portion and a fluorescence-emitting portion to prepare a mixture, a step S12 (hereinafter, also referred to as a “fluorescence fingerprint acquisition step”) of acquiring a fluorescence fingerprint for the mixture, and a step S13 (hereinafter, also referred to as an “evaluation step”) of evaluating a state of the composition based on the fluorescence fingerprint are performed.

In the present embodiment, a substance that nonspecifically acts on an object is used as the labeling substance. In the present specification, the expression “nonspecifically act” means that the labeling substance can act not only on one type of substance but also on a plurality of substances, or can act not only on one position of a specific substance but also on a plurality of positions thereof. In the present embodiment, the interaction portion of the labeling substance has a nucleic acid structure capable of interacting with each of a plurality of types of objects, or has a nucleic acid structure capable of interacting with a plurality of positions of each object.

As described above, in a method of analyzing a fluorescence signal, a labeling substance that specifically acts only on a substance to be measured has been conventionally used. On the other hand, intensive studies by the present inventors reveal that not only the presence or absence of an object but also the state of a composition can be evaluated by using a labeling substance that nonspecifically interacts with the object. For example, the molecular structure around the fluorescence-emitting portion of the labeling substance and the interaction state of the interaction portion are different between when the labeling substance interacts with a specific object and when the labeling substance interacts with a component changed from the specific object. Therefore, the fluorescence wavelength and the fluorescence intensity emitted from the fluorescence-emitting portion slightly change. In addition, in a case where the labeling substance is capable of interacting with a plurality of sites of the object, when the state of the object changes, the labeling substance does not interact with the site whose state has changed, or even when the labeling substance interacts with the site whose state has changed, the molecular structure around the site is different, so that the fluorescence wavelength and the fluorescence intensity slightly change. Therefore, if these differences can be detected, the state of the composition can be evaluated.

However, it is difficult to specify these differences by a conventional general method of analyzing fluorescence signals. Therefore, in the present embodiment, a fluorescence fingerprint is used, and the state of the composition is specified based on the fluorescence fingerprint. In the acquisition of the fluorescence fingerprint, the measurement is comprehensively performed while changing the wavelength of the excitation light and the wavelength of the fluorescence to be observed. Therefore, the change is easily detected, and the state of the composition can be specified in detail.

In the present embodiment, as shown in FIG. 2, the mixture preparation step S1, the fluorescence fingerprint acquisition step S12, and the evaluation step S13 may be performed once each. On the other hand, the mixture preparation step S11 and the fluorescence fingerprint acquisition step S12 may be performed a plurality of times by using a plurality of types of labeling substances and changing the type of the labeling substance or the combination of the labeling substances, and the evaluation step S13 may be performed based on a plurality of fluorescence fingerprints. The evaluation step S13 is preferably performed based on a plurality of fluorescence fingerprints because the state of the composition can be more accurately evaluated. Each step will be described below.

### (1) Mixture Preparation Step

In the mixture preparation step S11 of the present embodiment, a composition containing one or more types of objects and a labeling substance are mixed to prepare a mixture.

The composition used in the present step may contain one or more types of objects whose presence, concentration, degree of deterioration, and the like are desired to be specified by interaction of the labeling substance. The composition may contain components other than the object as long as the object and effect of the present embodiment are not impaired, and may contain, for example, a solvent.

The type of the object is not particularly limited as long as at least a part of the object has a structure capable of interacting with the interaction portion (nucleic acid structure) of the labeling substance. Examples of the object include nucleic acids such as RNA and DNA, proteins such as amyloid P, tartaric acid, polyphenol, and the like in wine, mycotoxins, microorganisms, fatty acids, and fibers (animal hairs and the like). Examples of the object also include those in which a part or all of the components are structurally changed, and degraded products and aggregates of the components.

For example, in the case of measuring the degree of deterioration of the mRNA vaccine, the deteriorated vaccine (composition) contains an object such as mRNA or a degraded product of mRNA.

On the other hand, the labeling substance to be mixed with the composition in this step may have an interaction portion including a nucleic acid structure capable of interacting with the object, and a fluorescence-emitting portion capable of emitting light by irradiation with excitation light. The labeling substance may also include structures in addition to these portions. The labeling substance may further have, for example, a nucleic acid structure (hereinafter, also referred to as “another nucleic acid structure”) that does not contribute to interaction with the object, a quenching portion for controlling fluorescence of the fluorescence-emitting portion, an artificial nucleic acid structure, and a photoresponsive group. In this step, only one type of labeling substance may be mixed with the composition, or two or more types of labeling substances may be combined and mixed.

The interaction portion of the labeling substance has a nucleic acid structure capable of nonspecifically interacting with the object. Examples of the interaction between the interaction portion and the object include a hydrogen bond (hybridization) between nucleic acids, a hydrogen bond with a protein amide group or an amino acid in a biological substance such as an enzyme or a tumor marker, a hydrogen bond with an acid, and an interaction due to the shape of the formed three-dimensional structure.

The nucleic acid structure of the interaction portion is appropriately selected according to the structure or the like of the object. When the object is a nucleic acid, the nucleic acid structure preferably has a base sequence complementary to a part of the base sequence of the object. In addition, the complementary sequence of the nucleic acid structure is preferably composed of a relatively short base sequence. When the nucleic acid structure (base sequence) is short, it becomes easy to interact not only with the nucleic acid structure of one type of object but also with the nucleic acid structures of a plurality of types of objects. In addition, when the object contains a plurality of the same or similar base sequences, such a short base sequence easily interacts with each of the base sequences. However, when the number of bases in the complementary sequence in the nucleic acid structure is excessively small, the nucleic acid structure acts on many structures of the object, so that the fluorescence fingerprint becomes very complicated. Therefore, the number of complementary bases in the nucleic acid structure of the interaction portion is preferably about 5 or more and 20 or less, and more preferably 7 or more and 10 or less. When the number of bases is in this range, the labeling substance is likely to moderately interact with the object. The labeling substance contains one or more such interaction portions (nucleic acid structures) in the molecule.

On the other hand, the structure and position of the fluorescence-emitting portion contained in the labeling substance are not particularly limited. For example, the fluorescence-emitting portion may be arranged at a position away from the interaction portion via another nucleic acid structure. However, the fluorescence-emitting portion is preferably arranged at a position close to the interaction portion, and is preferably arranged at an end of the interaction portion or between bases of the nucleic acid structure of the interaction portion. When the distance between the fluorescence-emitting portion and the interaction portion is short, the wavelength and intensity of the fluorescence emitted from the fluorescence-emitting portion are likely to change according to the interaction state between the interaction portion and the object or the like.

The number of fluorescence-emitting portions contained in the labeling substance is not particularly limited, and may be one or more. When the labeling substance has a plurality of fluorescence-emitting portions, it is possible to cause the fluorescence-emitting portions to act on each other and self-quench when the labeling substance does not interact with the object, or to increase the emission intensity when the labeling substance interacts with the object, for example.

The type of dye contained in the fluorescence-emitting portion is not particularly limited. Examples thereof include general fluorescent dyes (for example, cyanine dyes, merocyanine dyes, acridine dyes, coumarin dyes, ethidium dyes, flavin dyes, fused aromatic ring dyes, and xanthene dyes).

The labeling substance may also have an artificial nucleic acid structure between bases of the nucleic acid structure. The artificial nucleic acid structure refers to a nucleic acid structure obtained by introducing a non-natural portion into a natural nucleic acid, or synthesizing only a non-natural portion. The artificial nucleic acid structure is a fully artificially synthesized molecule that usually does not exist in nature. The artificial nucleic acid structure thus has a feature of being hardly recognized by a nucleolytic enzyme or the like present in the air and being hardly degraded. By inserting such an artificial nucleic acid structure into the interaction portion, it is possible to stabilize the labeling substance, and increase the fluorescence intensity of the fluorescence-emitting portion, for example. Examples of the artificial nucleic acid structure include, but are not limited to, acyclic threoninol nucleic acid (aTNA), serinol nucleic acid (SNA), peptide nucleic acid (PNA), glycol nucleic acid (GNA), and locked nucleic acid (LNA).

The labeling substance may also contain an insulator for increasing the fluorescence intensity of the fluorescence-emitting portion, between bases of the nucleic acid structure of the interaction portion. When the labeling substance contains an insulator, transfer of electrons to another fluorescence-emitting portion or the like is suppressed, and the fluorescence intensity of the fluorescence-emitting portion tends to increase. The insulator is usually arranged adjacent to the fluorescence-emitting portion. The insulator can be, for example, a cyclic body having a non-planar structure, and examples thereof include structures derived from cyclobutane, cyclopentane, cyclohexane, cycheptane, cyclopentene, cyclohexene, cycloheptene, and derivatives thereof.

The labeling substance may also have a photoresponsive group whose structure is changed by irradiation with light having a specific wavelength, between bases of the nucleic acid structure of the interaction portion. When there is a photoresponsive group between bases of the nucleic acid structure of the interaction portion, the interaction state between the labeling substance (interaction portion) and the object changes by irradiation with specific light. As a result, the wavelength and intensity of the fluorescence emitted from the fluorescence-emitting portion are more finely adjusted. Examples of the photoresponsive group include a group whose structure is changed from a planar structure to a three-dimensional structure by irradiation with specific light. Specifically, the photoresponsive group includes a group that changes from a trans-form to a cis-form, and a group that changes from a merocyanine-form to a spiropyran-form. In particular, a structure derived from azobenzene, spiropyran, stilbene, or a derivative thereof is preferable because the structure is reversibly changed by irradiation with light having a specific wavelength.

Here, the labeling substance may have a stem structure and a loop structure. When the labeling substance has a fluorescence-emitting portion and a quenching portion, the fluorescence-emitting portion and the quenching portion corresponding to the fluorescence-emitting portion can be arranged to face each other in the stem structure. That is, in a normal state, the quenching portion and the fluorescence-emitting portion interact with each other, to suppress the light emission of the fluorescence-emitting portion. On the other hand, when the labeling substance interacts with an object or the like, the stem structure is opened, the suppression of quenching by the quenching portion is canceled, and the fluorescence-emitting portion emits fluorescence. The quenching portion is not particularly limited as long as it can act on the fluorescence-emitting portion to suppress the light emission, and can have, for example, a structure derived from azobenzene or a derivative thereof.

When the labeling substance has a stem structure and a loop structure, the interaction portion may be arranged in the loop structure, may be arranged in the stem structure, or may be arranged over both of them.

For example, when the object is an RNA vaccine, a labeling substance or the like in which one strand of the stem structure is a poly U sequence, the other strand of the stem structure is a poly A sequence, and the loop structure is an interaction portion may be used. In such a labeling substance, a stem is formed by the poly U sequence and the poly A sequence in a normal state. On the other hand, when the labeling substance is mixed with the object, the terminal (for example, the poly A sequence) of the object is brought close to the labeling substance (here, the poly U sequence that is complementary), so that other complementary base sequence portions are also brought close to each other, and the stem is opened. Hybridization is likely to occur, leading to an improvement in efficiency. In addition, a labeling substance or the like in which one side of the stem structure is a fluorescence-emitting portion and the other side of the stem structure has a quenching portion may also be used. In a normal state, the fluorescence-emitting portion and the quenching portion are close to each other, to suppress the light emission of the fluorescence-emitting portion. On the other hand, when the labeling substance is mixed with the object, the stem is opened, so that the quenching portion is separated from the fluorescence-emitting portion, and the fluorescence-emitting portion can emit fluorescence.

The labeling substance may be directly mixed with the composition, or a labeling substance contained in particles such as gelatin nanoparticles and lipid nanoparticles may be mixed with the composition. When the labeling substance is contained in gelatin nanoparticles, lipid nanoparticles, or the like, the nucleic acid structural portion or the like in the labeling substance is less likely to be affected by an enzyme or the like in the air. Therefore, degradation of the labeling substance and the like can be prevented, stability is improved, and long-term storage is also possible. The labeling substance of the present embodiment, which nonspecifically acts, does not need to be adjusted for each measurement target, and thus can quickly respond to a new measurement target by preparing and storing a plurality of types of labeling substances in advance.

When the labeling substance is contained in gelatin nanoparticles, lipid nanoparticles, or the like, the labeling substance as is cannot normally interact with the object. Therefore, the labeling substance needs to be released from the particles. The labeling substance can be easily released by adding a surfactant or making the liquid property of the mixture acidic. In addition, when the object is an RNA vaccine, since the RNA vaccine is usually protected by lipid nanoparticles or the like as described above, the mRNA contained is similarly released and measured.

### (2) Fluorescence Fingerprint Acquisition Step

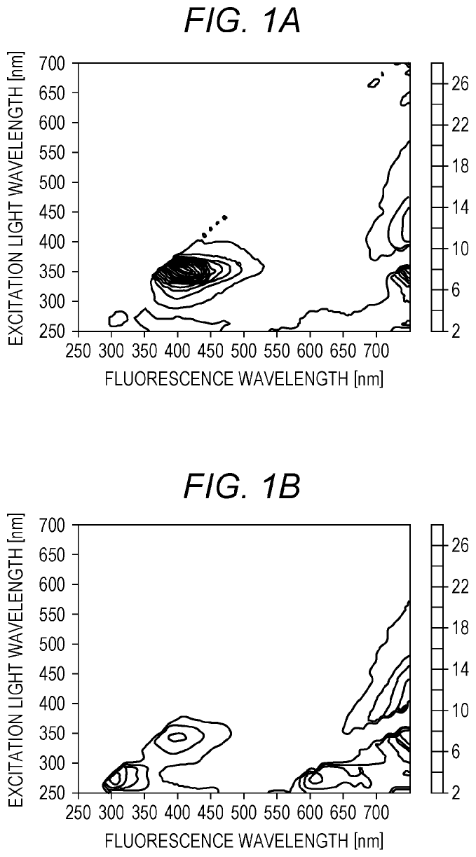

In the fluorescence fingerprint acquisition step S12, a fluorescence fingerprint is acquired from the above-described mixture. Usually, the fluorescence fingerprint is preferably three-dimensional data of the wavelength of the excitation light with which the mixture is irradiated, the wavelength of the fluorescence emitted from the mixture irradiated with the excitation light (the fluorescence emitting site), and the intensity thereof. The fluorescence fingerprint can be represented by graphs as shown in, for example, FIGS. 1A and 1B. FIG. 1A is a fluorescence fingerprint of a specific resin, and FIG. 1B is a fluorescence fingerprint after the resin is heated and modified.

Here, a method of acquiring the fluorescence fingerprint of the mixture is as follows. First, the mixture is irradiated with excitation light having a specific wavelength from an excitation light source. Then, the wavelength and intensity of fluorescence emitted from the mixture when the mixture is irradiated with the excitation light are measured. Next, the wavelength of the excitation light is shifted by a desired width (for example, 10 nm), and the wavelength and intensity of the fluorescence are measured in the same manner. These operations are repeated. As a result, data of the fluorescence wavelength and the fluorescence intensity corresponding to the excitation light in the desired wavelength range is obtained. Then, the obtained data is converted into three-dimensional data to thereby acquire a fluorescence fingerprint.

The wavelength of the excitation light used to acquire the fluorescence fingerprint is appropriately selected according to the type of the fluorescence-emitting portion of the labeling substance, the type of the object, and the like. For example, when the object is an RNA vaccine or the like, visible light can be used. The light source used for irradiation of the excitation light is not particularly limited, but may be a supercontinuum light source (broadband pulse light source that emits strong light with aligned phases over a very wide wavelength range by utilizing the non-linear effect of an optical fiber, and is also called “SC light source”) or an LED. With these light sources, the amount of light can be increased, and a good fluorescence fingerprint can be easily acquired. Note that a plurality of light sources may be combined.

On the other hand, the measurement of the wavelength and intensity of the fluorescence emitted from the mixture includes measurements with a spectrofluorometer and the like. The measurement may be performed using a plurality of spectrofluorometers.

The wavelength of the excitation light, the wavelength of the fluorescence, and the fluorescence intensity can be converted into three-dimensional data by, for example, a general personal computer.

### (3) Evaluation Step

In the evaluation step S13, the state of the composition is evaluated. The state of the composition in the present specification refers to the concentration of the object contained in the composition, the constitution of the composition, the alteration state or the deterioration state of a specific object, and the like. For example, in the evaluation step S13, the state of the composition includes the degree of deterioration, the degree of aggregation, and the like of the object, the degree of chemical or physical change of the object, the evaluation of the authenticity of the composition, the evaluation of the usability of the composition, and the like.

In particular, in the conventional method, it has been difficult to specify the degree of deterioration of the object. Therefore, it is particularly preferable to evaluate the degree of deterioration of the object. In general, when an object is deteriorated, it is often difficult to separate the object and the deteriorated product, or the difference is too small to be discriminated. On the other hand, in the method of the present embodiment, the minute difference can be detected, and evaluation can be qualitatively performed with high accuracy.

Here, in the evaluation of the state of the composition, the degree of state change may be specified by comparing the fluorescence fingerprint obtained in the above-described fluorescence fingerprint acquisition step S12 with the fluorescence fingerprint of the ideal state of the object (for example, the object that is not deteriorated). In addition, a fluorescence fingerprint after the state of the object has changed may be acquired and compared with the fluorescence fingerprint to specify the degree of state change. These comparisons may be performed by the above-described personal computer.

In addition, the fluorescence fingerprint obtained in the fluorescence fingerprint acquisition step S12 may be converted into lower dimensional data by statistical analysis processing, and may be parameterized so as to clearly express characteristics for each state of the composition to evaluate the composition. Examples of the statistical analysis processing method include multivariate analysis and data mining. Specific examples thereof include data structure analysis, discriminant analysis, pattern classification, multiway data analysis, regression analysis, and machine learning.

Examples of the data structure analysis include principal component analysis, factor analysis, correspondence analysis, and independent component analysis. Examples of the discriminant analysis include linear discriminant analysis and non-linear discriminant analysis. Examples of the linear discriminant analysis include canonical discriminant analysis, and examples of the non-linear discriminant analysis include a decision tree.

Examples of the pattern classification include cluster analysis and multidimensional scaling. Examples of the regression analysis include linear regression and non-linear regression. Here, examples of the linear discriminant analysis include a partial least square (PLS) regression, a single regression analysis, a multiple regression analysis, and a principal component regression, and examples of the non-linear discriminant analysis include a logistic regression and a regression tree.

Examples of the machine learning include a neural network, a self-organizing map, group learning, and a genetic algorithm. In the statistical analysis processing, any analysis method may be used as long as the method can most accurately analyze the state of the composition.

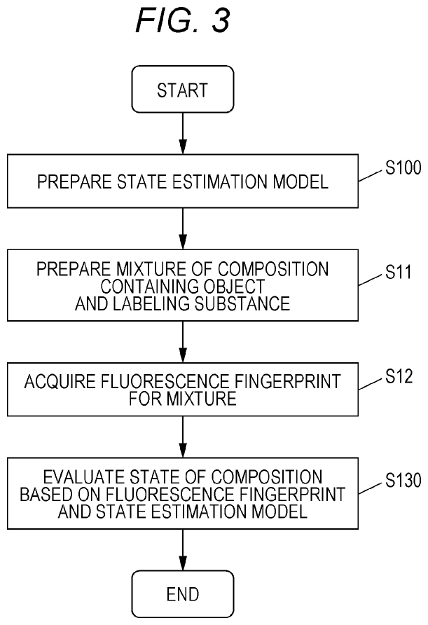

Furthermore, in this step, as in the modification shown in the flow of FIG. 3, when the state of the composition is evaluated, a step S100 of preparing a state estimation model in advance may be performed, and then a step S130 of evaluating the state of an object with respect to the fluorescence fingerprint obtained in the fluorescence fingerprint acquisition step S12 may be performed. Evaluation can be easily performed by using the state estimation model. Note that a state estimation model created in advance can be used, and the state estimation model does not need to be created every time. Furthermore, in the flow of FIG. 3, the step S100 of preparing the state estimation model is described before the mixture preparation step S11, but the step S100 of preparing the state estimation model may be performed before or after the fluorescence fingerprint acquisition step S12.

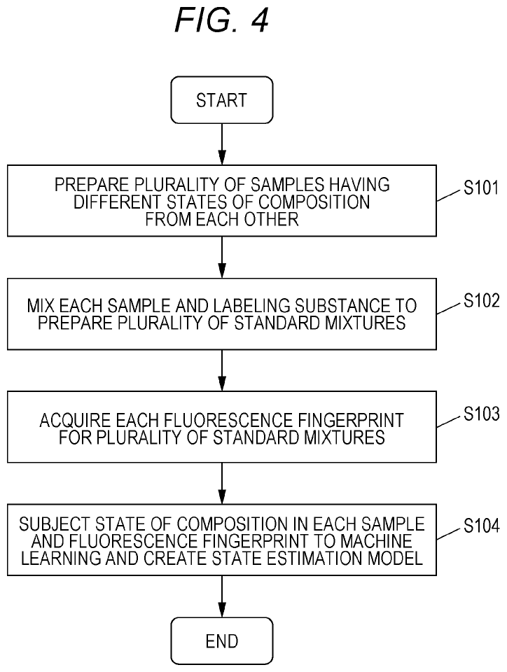

The state estimation model can be created, for example, as follows. A creation flow of the state estimation model is illustrated in FIG. 4. First, a plurality of samples having different states of the composition from each other are prepared (S101). At this time, when it is desired to finely evaluate the deterioration rate of the object or the like, a plurality of samples in which the deterioration rate of the object in the composition has been adjusted may be prepared. On the other hand, when it is difficult to finely adjust the state of the composition and qualitative evaluation is performed, a sample having a large state change, a sample having a moderate state change, a sample having a small state change, and the like may be prepared.

Then, similarly to the above-described mixture preparation step, a labeling substance is mixed with each of a plurality of samples having different composition states to prepare a standard mixture (S102). Then, a standard fluorescence fingerprint is acquired for each of the plurality of standard mixtures (S103). A method for acquiring the standard fluorescence fingerprint is similar to the above-described fluorescence fingerprint acquisition step. Thereafter, the obtained standard fluorescence fingerprint and the state of the composition are subjected to machine learning to obtain a state estimation model (S104). When the state estimation model is created, it is preferable to acquire many standard fluorescence fingerprints using not only one type of labeling substance (or one combination of labeling substances) but also different types of labeling substances. For example, in a case where there are 5 samples and 10 types of labeling substances, 5×10 types of fluorescence fingerprints are subjected to machine learning.

As a method of performing machine learning, a known method can be used. For example, multivariate analysis is performed on a specific sample with a standard fluorescence fingerprint set as an explanatory variable and a degree of state change set as an objective variable to obtain a similarity index. The similarity index can be selected from cosine similarity, Pearson correlation coefficient, deviation pattern similarity, Euclidean distance similarity, Morisita similarity index, standard Euclidean distance similarity, Mahalanobis' distance similarity, Manhattan distance similarity, Chebyshev distance similarity, Minkowski distance similarity, Jaccard coefficient similarity, Dice coefficient similarity, Simpson coefficient similarity, and the like.

In the similarity index calculation step, a similarity index of a fluorescence fingerprint is calculated for a sample having a certain degree of state change prepared in advance, on the assumption that there is a correlation between the degree of state change as an objective variable and the similarity index of the fluorescence fingerprint of the object to be measured. This is performed on a plurality of samples having different degrees of state change, and optimization is repeatedly performed so as to reduce an error between the calculated similarity index and the degree of state change of the prepared sample, whereby an estimation model can be created.

Many analysis methods have been developed for two-dimensional data. On the other hand, the above-described fluorescence fingerprint is three-dimensional data of the wavelength of the excitation light, the wavelength of the fluorescence, and the fluorescence intensity. Therefore, the three-dimensional data may be developed into two-dimensional data, and multivariate analysis may be performed. For example, the result of fluorescence fingerprint measurement may be developed into two-dimensional data of wavelength conditions (combination of excitation wavelength and fluorescence wavelength) and fluorescence intensity for each labeling substance, and multivariate analysis may be performed. In addition, processing such as meancentering, normalization, autoscale, second derivative, baseline correction, and smoothing may be performed on the two-dimensionally developed fluorescence fingerprint information. As a result, it is possible to emphasize information included in each piece of data and match a scale of data of different samples. On the other hand, multivariate analysis may be performed using three-dimensional data as is.

It is also possible to detect, as necessary from the obtained similarity indices, a marker signal (combination of excitation wavelength and fluorescence, and the like) that is important for estimation (change is significant depending on the state of the composition). At this time, principal component regression, cluster analysis, discriminant analysis, SIMCA, multiple regression analysis, PLS regression analysis, PLS discrimination, SVM regression, SVM discrimination, RF regression, and/or RF discrimination may be performed as multivariate analysis to detect a marker signal. In addition, the marker signal may be detected based on one or more indices of a regression coefficient obtained by multivariate analysis, a factor loading, loading, a selectivity ratio, variable importance in projection, variable importance, and out-of-bag error, which indicate the contribution ratio to regression/discrimination.

Then, the numerical value of the marker signal is estimated for a sample of a composition different from the sample for which the similarity index has been obtained. Thereafter, the data calculated by the estimation is compared with the actual data, and the optimization is repeatedly performed so as to reduce these errors. Through these operations, a state estimation model capable of specifying the state of the composition may be obtained from fluorescence fingerprint data or the like.

Then, the state of the composition can be evaluated by applying the fluorescence fingerprint obtained in the fluorescence fingerprint acquisition step to the state estimation model.

### (Effects)

In the present embodiment, a labeling substance that nonspecifically interacts with an object is used, and a fluorescence fingerprint is further acquired. According to the method of the present embodiment, the state of the composition can be grasped by a simple method without separating the object or the like in the composition. In addition, in the method, even when there is a plurality of objects, it is not necessary to prepare a labeling substance for each object, so that the cost and time required for preparation of the labeling substance are small. Furthermore, even when the object changes to an unexpected structure, there is a high possibility that the object can be detected by the labeling substance. Therefore, for example, an mRNA vaccine or the like can be evaluated and specified by a very simple process.

### 2. Second Embodiment

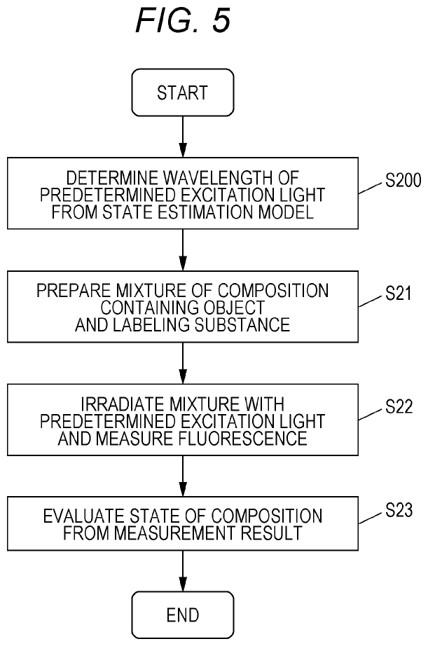

The flow of the composition evaluation method of the present embodiment is shown in FIG. 5. In the composition evaluation method of the present embodiment, a step S21 (hereinafter, also referred to as a “mixture preparation step”) of mixing a composition containing one or more types objects and a labeling substance having an interaction portion and a fluorescence-emitting portion to prepare a mixture, a step S22 (hereinafter, also referred to as a “fluorescence measurement step”) of irradiating the mixture with predetermined excitation light and measuring fluorescence, and a step S23 (hereinafter, also referred to as an “evaluation step”) of evaluating a state of the composition from a result of the measurement are performed. Also in the present embodiment, as in the first embodiment, a substance that nonspecifically acts on an object is used as the labeling substance.

The wavelength of the excitation light to be emitted in the fluorescence measurement step of the present embodiment is determined in advance by performing, for example, a step (S200) of determining a predetermined wavelength from the state estimation model. That is, in the present embodiment, how the emission wavelength, emission intensity, and the like of the mixture change when the state of the composition changes is analyzed in advance, and then the wavelength of the excitation light suitable for detecting the change in the state is selected. Therefore, in the fluorescence measurement step, the state of the composition can be easily evaluated by measuring the fluorescence wavelength and the fluorescence intensity generated by the excitation light having a predetermined wavelength, and comparing these with reference values or the like. In the flow illustrated in FIG. 5, the step S200 of determining the wavelength of the excitation light is described before the mixture preparation step S21. However, once the step S200 of determining the wavelength of the excitation light is performed, it is not necessary to perform the step S200 of determining the wavelength every time. Therefore, if the wavelength of the excitation light has already been determined, only the mixture preparation step S21, the fluorescence measurement step S22, and the evaluation step S23 are performed. Since the mixture preparation step S21 of the present embodiment is similar to the mixture preparation step S11 of the first embodiment described above, the fluorescence measurement step and the evaluation step will be described.

### (1) Fluorescence Measurement Step

In the fluorescence measurement step S22, the mixture obtained in the mixture preparation step S21 is irradiated with predetermined excitation light, and fluorescence emitted from the mixture is measured. The excitation light having a predetermined wavelength to be emitted in this step is an excitation light specified by creating the state estimation model as described above. In this step, only one type of excitation light may be emitted, and then the fluorescence emitted from the mixture may be measured. It is preferable to sequentially emit excitation light having a plurality of wavelengths and measure the fluorescence emitted from the mixture for each of the excitation light from the viewpoint of enabling more accurate evaluation.

Note that the light source of the excitation light in this step is appropriately selected according to the wavelength of the excitation light, and the light source can be an SC light source, an LED, or the like. The fluorescence measuring device may be, for example, a spectrofluorometer or a one-dimensional line sensor.

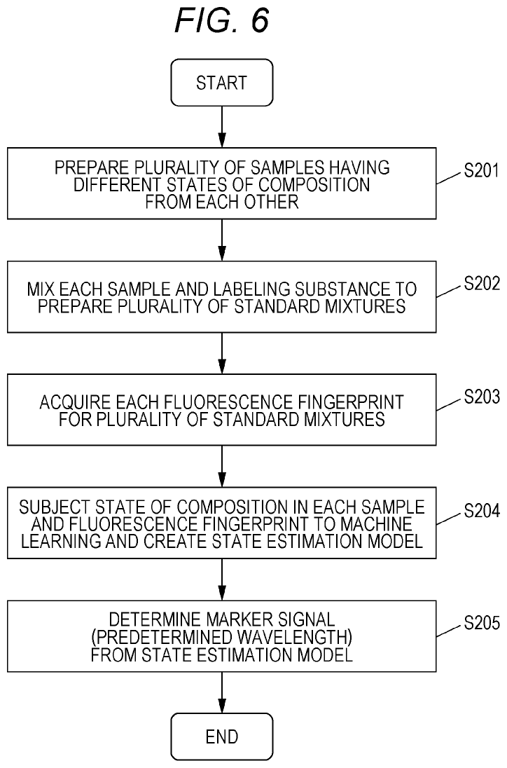

Here, a method of creating a state estimation model for determining the wavelength of the excitation light with which the mixture is irradiated in this step is substantially the same as the method described in the evaluation step S13 of the first embodiment described above. FIG. 6 illustrates a flow for determining the wavelength of the excitation light. First, a plurality of samples having different states of the composition are prepared (S201). Then, similarly to the above-described mixture preparation step S21, a labeling substance is mixed with each of the plurality of samples to prepare a standard mixture (S202). Then, a standard fluorescence fingerprint is acquired for each of the plurality of standard mixtures (S203). Then, the obtained standard fluorescence fingerprint and the state of the composition are subjected to machine learning (S204). Thereafter, one or more marker signals (for example, a combination of excitation wavelength and fluorescence wavelength) that are important for estimation (change is significant depending on the state change of the composition) are detected from the similarity indices calculated based on the standard fluorescence fingerprint and the state of the composition (S205). Then, one or two or more wavelengths of the excitation light in the marker signal are selected as the wavelength of the excitation light in the fluorescence measurement step S22.

### (2) Evaluation Step

In the evaluation step S23, the state of the composition is evaluated based on the data obtained in the fluorescence measurement step S22 described above. For example, from the state estimation model, pieces of information are acquired in advance, such as information indicating that when fluorescence at the wavelength λ2 is detected at the excitation wavelength λ1, the degree of deterioration of the object in the composition is high, and information indicating that when the intensity of the fluorescence wavelength λ4 is high at the excitation wavelength χ3, the composition is highly deteriorated. Then, the data of the wavelength and intensity of fluorescence measured in the fluorescence measurement step can be compared with these pieces of information to evaluate the state of the composition. In addition, the data obtained in the fluorescence measurement step S22 may be applied to the state estimation model created at the time of determining the predetermined wavelength described above to evaluate the state of the composition.

### (3) Effects

In the present embodiment, a labeling substance that nonspecifically interacts with an object is used. Furthermore, fluorescence measurement is performed by emitting an excitation wavelength determined in advance based on the state estimation model. Therefore, in the present embodiment, it is not necessary to measure the entire wavelength range at the time of measurement, and the state of the target composition can be easily evaluated by short-time measurement.

In addition, in the method, it is not necessary to prepare a labeling substance for each object, so that the cost and time required for preparation of the labeling substance are small. Furthermore, even when the object changes to an unexpected structure, there is a high possibility that the object can be detected by the labeling substance. Therefore, for example, an mRNA vaccine or the like can be evaluated and specified by a very simple process.

### 3. Others

In any of the above-described embodiments, the method for mixing the composition and the labeling substance in the mixture preparation step and the like is not particularly limited. For example, a plate having a plurality of recesses and containing different labeling substances in each of the recesses may be prepared, and a predetermined amount of the composition may be injected into each of the recesses by an inkjet method. Then, the fluorescence fingerprint acquisition step and the fluorescence measurement step may be performed using the plate. According to this method, a plurality of mixtures or standard mixtures can be easily prepared.

The present application claims priority based on Japanese Patent Application No. 2021-126668 filed on Aug. 2, 2021. Contents described in the application specification and drawings are all incorporated herein by reference.

### INDUSTRIAL APPLICABILITY

In the composition evaluation method of the present invention, it is possible to grasp the state of the composition by a simple method without separating an object. Therefore, the composition evaluation method of the present invention is useful for measuring the degree of deterioration of various medicines and foods, inspection in various medical fields, and the like.

1. A composition evaluation method including:
mixing a composition containing one or more types of objects and a labeling substance having an interaction portion and a fluorescence-emitting portion to prepare a mixture;
acquiring a fluorescence fingerprint for the mixture; and
evaluating a state of the composition based on the fluorescence fingerprint, wherein
the interaction portion has a nucleic acid structure capable of interacting with each of a plurality of types of the objects and/or a nucleic acid structure capable of interacting with a plurality of positions of each of one or more types of the objects.

2. The composition evaluation method according to claim 1, wherein
a plurality of types of the labeling substances having the interaction portions different from each other.

3. The composition evaluation method according to claim 1, wherein
in the evaluating a state, a degree of deterioration of the composition is evaluated.

4. The composition evaluation method according to claim 1, wherein
in the evaluating a state, the state of the object is evaluated based on a state estimation model, and
the state estimation model is created by
preparing a plurality of samples having different states of the composition from each other,
mixing the labeling substance with each of the plurality of samples to prepare a plurality of standard mixtures,
acquiring a standard fluorescence fingerprint for each of the plurality of standard mixtures, and
subjecting a state of the composition in each of the plurality of samples and each of the standard fluorescence fingerprints to machine learning.

5. The composition evaluation method according to claim 1, wherein
the interaction portion includes an artificial nucleic acid structure.

6. The composition evaluation method according to claim 1, wherein the labeling substance has the fluorescence-emitting portion between bases of the interaction portion.

7. The composition evaluation method according to claim 6, wherein
the labeling substance has a loop structure and a stem structure, and
in the stem structure, the fluorescence-emitting portion and a quenching portion corresponding to the fluorescence-emitting portion are arranged to face each other.

8. The composition evaluation method according to claim 1, wherein
the interaction portion has a photoresponsive group whose structure is changed by irradiation with light having a specific wavelength between bases of the nucleic acid structure.

9. The composition evaluation method according to claim 1, wherein
the labeling substance is contained in a particle.

10. A composition evaluation method including:
mixing a composition containing one or more types of objects and a labeling substance having an interaction portion and a fluorescence-emitting portion to prepare a mixture;
irradiating the mixture with excitation light having a predetermined wavelength and measuring fluorescence emitted from the mixture; and
evaluating a state of the object from a result of the measurement, wherein
the interaction portion of the labeling substance has a nucleic acid structure capable of interacting with each of a plurality of types of the objects and/or has a nucleic acid structure capable of interacting with a plurality of positions of each of the objects, and
a wavelength of the excitation light is
specified from a state estimation model created by
preparing a plurality of samples having different states of the composition from each other,
mixing the labeling substance with each of the plurality of samples to prepare a plurality of standard mixtures,
acquiring a standard fluorescence fingerprint for each of the plurality of standard mixtures, and
subjecting a state of the composition in each of the plurality of samples and each of the standard fluorescence fingerprints to machine learning.

11. The composition evaluation method according to claim 2, wherein
in the evaluating a state, a degree of deterioration of the composition is evaluated.

12. The composition evaluation method according to claim 2, wherein
in the evaluating a state, the state of the object is evaluated based on a state estimation model, and
the state estimation model is created by
preparing a plurality of samples having different states of the composition from each other,
mixing the labeling substance with each of the plurality of samples to prepare a plurality of standard mixtures,
acquiring a standard fluorescence fingerprint for each of the plurality of standard mixtures, and
subjecting a state of the composition in each of the plurality of samples and each of the standard fluorescence fingerprints to machine learning.

13. The composition evaluation method according to claim 2, wherein
the interaction portion includes an artificial nucleic acid structure.

14. The composition evaluation method according to claim 2, wherein the labeling substance has the fluorescence-emitting portion between bases of the interaction portion.

15. The composition evaluation method according to claim 2, wherein
the interaction portion has a photoresponsive group whose structure is changed by irradiation with light having a specific wavelength between bases of the nucleic acid structure.

16. The composition evaluation method according to claim 2, wherein
the labeling substance is contained in a particle.

17. The composition evaluation method according to claim 3, wherein
in the evaluating a state, the state of the object is evaluated based on a state estimation model, and
the state estimation model is created by
preparing a plurality of samples having different states of the composition from each other,
mixing the labeling substance with each of the plurality of samples to prepare a plurality of standard mixtures,
acquiring a standard fluorescence fingerprint for each of the plurality of standard mixtures, and
subjecting a state of the composition in each of the plurality of samples and each of the standard fluorescence fingerprints to machine learning.

18. The composition evaluation method according to claim 3, wherein
the interaction portion includes an artificial nucleic acid structure.

19. The composition evaluation method according to claim 3, wherein the labeling substance has the fluorescence-emitting portion between bases of the interaction portion.

20. The composition evaluation method according to claim 3, wherein
the interaction portion has a photoresponsive group whose structure is changed by irradiation with light having a specific wavelength between bases of the nucleic acid structure.
